(19) United States
(12) Patent Application Publication
(10) Pub. No.: US 2024/0066125 A1
(43) Pub. Date: Feb. 29, 2024

(54) GLOBO SERIES ANTIGENS-BINDING CHIMERIC ANTIGEN RECEPTORS AND USES THEREOF

(71) Applicant: OBI PHARMA, INC., TAIPEI (TW)

(72) Inventors: MING-TAIN LAI, TAIPEI (TW); JIANN-SHIUN LAI, TAIPEI (TW); SHIOU-LING JIAN, TAIPEI (TW); JHANG-SIAN YU, TAIPEI (TW); WOAN-ENG CHAN, TAIPEI (TW)

(21) Appl. No.: 18/259,277

(22) PCT Filed: Feb. 9, 2022
(86) PCT No.: PCT/US22/15720

§ 371 Date: Jun. 26, 2023

Related U.S. Application Data
(60) Provisional application No. 63/147,237, filed on Feb. 9, 2021.
(60) Provisional application No. 63/147,441, filed on Feb. 9, 2021.

(51) Int. Cl. A61K 39/00; A61K 35/17; A61P 35/00; C07K 16/30; C12N 5/0783

(52) U.S. Cl. CPC A61K 39/4631; A61K 35/17; A61K 39/4611; A61P 35/00; C07K 16/30; C12N 5/0636; C07K 2317/53; C07K 2317/622; C12N 2510/00

(57) ABSTRACT

The present disclosure relates to chimeric antigen receptors (CARs), which bind to Globo series antigens (e.g. Globo H, SSEA-3 or SSEA-4), including an antigen-binding fragment (Fab) or a single-chain variable fragment (scFv). Further, the present methods are also provided for administering CARs to a subject in an amount effective to inhibit cancer cells.

### CROSS-REFERENCE TO RELATED APPLICATIONS

This application claims priority of Provisional Application No. 63/147,237, filed on Feb. 9, 2021, and Provisional Application No. 63/147,441, filed on Feb. 9, 2021, the content of which are incorporated herein in its entirety by reference.

### FIELD OF THE INVENTION

The present invention relates to chimeric antigen receptors (CARs) which bind to Globo series antigens (e.g. Globo H, SSEA-3 or SSEA-4), including an antigen-binding fragment (Fab) or a single-chain variable fragment (scFv). Further, the present invention methods are also provided for administering CARs to a subject in an amount effective to inhibit cancer cells.

### SEQUENCE LISTING

The instant application contains a Sequence Listing which has been submitted electronically in ASCII format and is hereby incorporated by reference in its entirety. Said ASCII copy, created Jan. 13, 2022, is named “OBIP-4PCT_SEQList_ST25.txt” and is 53,248 bytes in size.

### BACKGROUND OF THE INVENTION

Numerous surface carbohydrates are expressed in malignant tumor cells. For example, Globo H (Fuc α1-->2Galß1-->3GalNAcß1-->3Gal α1-->4Galß1-->4Glc) has been shown to be overexpressed on a variety of epithelial cancers and is associated with tumor aggressiveness and poor prognosis in breast cancer and small cell lung carcinoma. Previous studies have shown that Globo H and stage-specific embryonic antigen 3 (Gal β1→3GalNAc, β1→3Gal α1→4Gal β1-4Glc β1) (SSEA-3, also called Gb5) were observed on breast cancer cells and breast cancer stem cells (Chang W W et al., (2008) PNAS, 105(33):11667-11672; Cheung S K et al., (2016) PNAS, 113(4):960-965). In addition, SSEA-4 (stage-specific embryonic antigen-4) (Neu5Ac α2→3Gal β1→3GalNAc β1→3Gal α1→4Gal β1→4Glcβ1) has been commonly used as a cell surface marker for pluripotent human embryonic stem cells and has been used to isolate mesenchymal stem cells and enrich neural progenitor cells (Kannagi R et al., (1983) EMBO J, 2:2355-2361). These findings support that Globo series antigens (Globo H, SSEA-3, and SSEA-4) are unique targets for cancer therapies and can be used to direct therapeutic agents in targeting cancer cells effectively.

Chimeric antigen receptors (CARs) are receptor proteins that have been engineered to give T cells the new ability to target a specific protein. CARs are synthetic receptors that redirect the specificity, function, and metabolism of T cells. CARs consist of a T-cell activating domain (typically including the zeta chain of the CD3 complex) and extracellular immunoglobulin-derived heavy and light chains to direct specificity (June C H and Sadelain M., (2018) N Engl J Med, 379(1):64-73). Introduction of CAR into T cells enables these effector cells to recognize tumor associated antigens (TAAs) via the single chain variable fragment (scFv) and activate T cells through the cytoplasmic signaling domains, releasing perforin, granzyme and various cytokines to exert potent anti-tumor effect. Thus, CAR T cells function in non-MHC restricted manner, which cleverly combine the potent tumor-killing capacity of cytotoxic T cells and the specific antigen recognition of antibody together. Compared with monoclonal antibody (mAb) therapy, CAR T cells approach is more effective in generating durable tumor response and also providing stronger penetrability in solid tumors with lower risk of resistance (Han Y et al., (2018) Am J Cancer Res, 8(1):106-119).

These findings support a rationale for the development of CARs Globo series antigens, as there is still an unmet need for effective treatment and/or prevention for cancer. The present invention provides CARs to Globo series antigens to satisfy these and other needs.

### SUMMARY OF THE INVENTION

The present invention relates to chimeric antigen receptors (CARs), which includes (1) a first endodomain and a single-chain variable fragment (scFv), or (2) a first endodomain and an antigen-binding fragment (Fab). Further, the first endodomain includes CD3zeta or Fc·RIγ, and the scFv or Fab recognizes Globo series antigens. Additionally, the CAR includes an amino acid sequence with 80% to 100% sequence identity to SEQ ID No: 14, 16 or 18 when the CARs include the first endodomain and scFv, and the CAR has an amino acid sequence with 80% to 100% sequence identity to SEQ ID No: 13, 15 or 17 when the CARs include the first endodomain and Fab.

In certain embodiment, the present invention provides for a chimeric antigen receptor (CAR) including a single chain Fv (scFv) that specifically binds to Globo H having an amino acid sequence that is with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or about 100% sequence identity to SEQ ID No: 14, 16 or 18.

In certain embodiment, the present invention provides for a chimeric antigen receptor (CAR) including an antigen-binding fragment (Fab) that specifically binds to Globo H having an amino acid sequence that is with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or about 100% sequence identity to SEQ ID No: 13, 15 or 17.

In certain embodiment, the CAR further includes a second endodomain including CD28, CD137, CD4, OX40, 4-1BB, CD3Z, or ICOS, wherein the scFv is fused to the second endodomain, and the second endodomain is fused to the first endodomain.

In certain embodiment, the scFv includes an amino acid sequence that is with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or about 100% sequence identity to SEQ ID No: 3 or 6.

In certain embodiments, the Chimeric antigen receptors (CARs) include a variable heavy chain region (V H) having an amino acid sequence that is with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or about 100% sequence identity to SEQ ID No: 1 or 4.

In certain embodiments, the Chimeric antigen receptors (CARs) include a variable light chain region (V_{L}) having an amino acid sequence that is with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or about 100% sequence identity to SEQ ID No: 2 or 5.

In certain embodiments, the Chimeric antigen receptors (CARs) include a hinge region having an amino acid sequence that is with at least about 90%, at least about 91%, at least about 92%, at least about 93%, at least about 94%, at least about 95%, at least about 96%, at least about 97%, at least about 98%, at least about 99% or about 100% sequence identity to SEQ ID No: 7.

In certain embodiments, the hinge region is CD8.

In certain embodiments, the Chimeric antigen receptors (CARs) include a CD28 region having an amino acid sequence that is with at least about 90%, at least about 91%, at least about 92%, at least about 93%, at least about 94%, at least about 95%, at least about 96%, at least about 97%, at least about 98%, at least about 99% or about 100% sequence identity to SEQ ID No: 8.

In certain embodiments, the Chimeric antigen receptors (CARs) include a 4-1BB region hinge having an amino acid sequence that is with at least about 90%, at least about 91%, at least about 92%, at least about 93%, at least about 94%, at least about 95%, at least about 96%, at least about 97%, at least about 98%, at least about 99% or about 100% sequence identity to SEQ ID No: 9.

In certain embodiments, the Chimeric antigen receptors (CARs) include a CD3zeta (CD3z) region having an amino acid sequence that is with at least about 90%, at least about 91%, at least about 92%, at least about 93%, at least about 94%, at least about 95%, at least about 96%, at least about 97%, at least about 98%, at least about 99% or about 100% sequence identity to SEQ ID No: 10 or 11.

In certain embodiments, the CAR binds to Globo series antigens. Further, the Globo series antigens selected from the group consisting of Globo H, stage-specific embryonic antigen 3 (SSEA-3), and stage-specific embryonic antigen 4 (SSEA-4).

Further, the present invention also provides a method for treating a subject with a tumor, and the method includes:
(A) obtaining T cells from the subject having the tumor;
(B) generating chimeric antigen receptor expression T cells (CAR-T cells) by transducing the T cells with a vector including a nucleic acid sequence encoding a chimeric antigen receptor (CAR);
(C) expanding the CAR-T cells; and
(D) infusing the expanded CAR-T cells into the subject, whereby an immune response is raised.
   Further, the CAR includes a single-chain variable fragment (scFv) or an antigen-binding fragment (Fab). The scFv or Fab recognizes Globo series antigens. Further, the CAR includes an amino acid sequence with 80% to 100% sequence identity to SEQ ID No: 14, 16 or 18 when the CARs include the scFv, and the CAR has an amino acid sequence with 80% to 100% sequence identity to SEQ ID No: 13, 15 or 17 when the CARs include the Fab.

In certain embodiment, the subject is human.

In certain embodiment, the immune response is mediated by T cells.

In certain embodiment, the vector includes a lentivirus, a gamma retrovirus, or an adeno-associated vims.

In certain embodiment, cancers expressing Globo series antigens (e.g. Globo H) include, but are not limited to, sarcoma, skin cancer, leukemia, lymphoma, brain cancer, glioblastoma, lung cancer, breast cancer, oral cancer, head-and-neck cancer, nasopharyngeal cancer, esophagus cancer, stomach cancer, liver cancer, bile duct cancer, gallbladder cancer, bladder cancer, pancreatic cancer, intestinal cancer, colorectal cancer, kidney cancer, cervix cancer, endometrial cancer, ovarian cancer, buccal cancer, oropharyngeal cancer, laryngeal cancer, esophageal cancer, rectal cancer, biliary cancer, cervical cancer, testicular cancer, neuroendocrine cancer, adrenal cancer, thyroid cancer, bone cancer, basal cell carcinoma, squamous cell carcinoma, melanoma and prostate cancer.

In certain embodiment, the exemplary 2C2 (Anti-Globo H monoclonal antibody) is as described in PCT patent publications (WO2015157629A2 and WO2017062792A1), the contents of which are incorporated by reference in its entirety.

In certain embodiment, the exemplary R783 (Anti-Globo H monoclonal antibody) is as described in U.S. provisional patent applications No. 63/147,441.

The present invention also provides for a method of inhibiting Globo H expressing cancer cells, comprising administering to a subject in need thereof an effective amount of the antibody or antigen-binding portion thereof described herein, wherein the Globo H expressing cancer cells are inhibited.

The present invention also encompasses several amino acid sequences of Chimeric antigen receptors (CARs), including an antigen-binding fragment (Fab) or a single-chain variable fragment (scFv) that specifically binds to a carbohydrate antigen such as Globo series antigens. In one embodiment, the carbohydrate antigen is SSEA-3. In another embodiment, the carbohydrate antigen is SSEA-4. In yet another embodiment, the carbohydrate antigen is Globo H.

The present Chimeric antigen receptors (CARs) and methods can be used in all vertebrates, e.g., mammals and non-mammals, including human, mice, rats, guinea pigs, hamsters, dogs, cats, cows, horses, goats, sheep, pigs, monkeys, apes, gorillas, chimpanzees, rabbits, ducks, geese, chickens, amphibians, reptiles and other animals.

### BRIEF DESCRIPTION OF DRAWINGS

FIG. 1. The schematic diagram of Globo-H CAR. There are total six Globo H CAR constructs [2C2-Fab CAR: SEQ ID No. 13; 2C2-scFv CAR: SEQ ID No. 14; R783-Fab CAR: SEQ ID No. 15; R783-scFv CAR: SEQ ID No. 16; 2C2-Fab CAR (CD3zeta mutant): SEQ ID No. 17; R783-scFv CAR (CD3zeta mutant: SEQ ID No. 18].

FIG. 2. In vitro cytotoxicity of Globo H CAR T cells. (A) MCF-7: breast cancer cell line-Globo H positive, (B) HCC-1428: breast cancer cell line-Globo H positive, (C) NCI-N87: gastric cancer cell line-Globo H positive, (D) SW-480: colon cancer cell line-Globo H positive, (E) SK-OV-3: ovarian cancer cell line-Globo H negative.

FIG. 3. In vitro cytotoxicity of Globo H CAR T cells with CD3zeta mutant (CD3zm). (A) HCC-1428: breast cancer cell line-Globo H positive, (B) MCF-7: breast cancer cell line-Globo H positive, (C) NCI-N87: gastric cancer cell line-Globo H positive, (D) SK-OV-3: ovarian cancer cell line-Globo H negative.

FIG. 4. In vitro persistence of Globo H CAR T cells with CD3zeta mutant (CD3zm). (A) Cytotoxicity between MCF-7 (Globo H positive) and SK-OV-3 (Globo H negative) cancer cell lines. (B) T cell number between MCF-7 (Globo H positive) and SK-OV-3 (Globo H negative) cancer cell lines.

FIG. 5. In vivo efficacy of Globo H CAR T cells in NCI-N87 gastric xenograft model. (A) Tumor bioluminescence images from Day 11 to Day 32. (B) Kinetics of tumor bioluminescence.

FIG. 6. In vivo efficacy of Globo H CAR T in different tumor models. (A) MCF-7 breast cancer orthotopic model, (B) HCC-1428 breast cancer orthotopic model, (C) SW480 colon cancer xenograft model.

FIG. 7. In vivo persistence of Globo H CAR T in NCI-N87 gastric xenograft model. (A) Kinetics of tumor bioluminescence in four tumor groups (1^{st} tumor growth) and one tumor-free mock control from Day 0 to Day 47. The curves show data for mean±SD of three mice per group. The primary tumors treated with 2C2-Fab CAR T or 2C2-Fab (CD3zm) CAR T cells have been all eliminated at Day 25, (B) Kinetics of 2^{nd} and 3^{rd} tumor bioluminescence in 2C2-Fab and 2C2-Fab (CD3zm) CAR T cell therapeutic groups from Day 45 to Day 90.

### DETAILED DESCRIPTION OF THE INVENTION

As used herein, the articles “a” and “an” refer to one or more than one (i.e., at least one) of the grammatical object of the article. By way of example, “an element” means one element or more than one element

An “effective amount,” as used herein, refers to a dose of the vaccine or pharmaceutical composition that is sufficient to reduce the symptoms and signs of cancer, such as weight loss, pain and palpable mass, which is detectable, either clinically as a palpable mass or radiologically through various imaging means. The term “effective amount” and “therapeutically effective amount” are used interchangeably.

The term “subject” can refer to a vertebrate having cancer or to a vertebrate deemed to be in need of cancer treatment. Subjects include all warm-blooded animals, such as mammals, such as a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, mouse, rabbit, rat, gerbil, guinea pig, etc.). Thus, veterinary uses and medical formulations are contemplated herein.

The following examples of specific aspects for carrying out the present invention are offered for illustrative purposes only and are not intended to limit the scope of the present invention in any way.

### EXAMPLES

### Example 1. Globo H CAR-T Cells Preparation

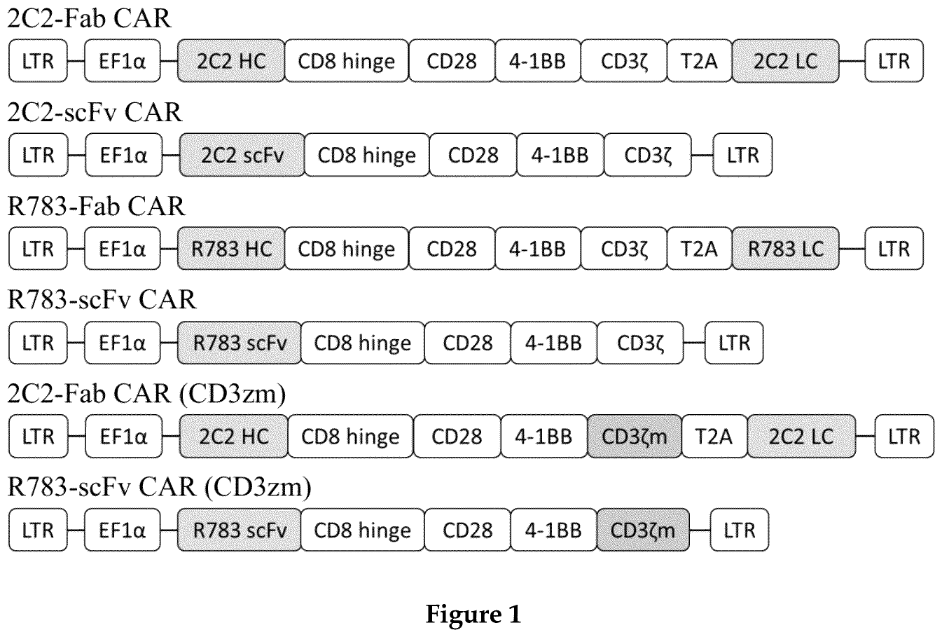

Anti-Globo H antibody 2C2 or R783 were used to perform scFv or Fab CAR constructs. Intracellular domains contain CD28, 4-1BB and CD3zeta to perform third generation CAR. CD3zeta mutant was a mutant domain with less tyrosine kinase activity to generate persistence CAR. Schematic diagram of Globo-H CAR was listed in FIG. 1. Human T cells isolate from cryopreserved human PBMCs by human Pan T Cell Isolation Kit (Miltenyi Biotec, Cat. No. 130-096-535). Activate and expand pan T cells with Dynabeads Human T-Activator CD3/CD28 (ThemoFisher, Cat. No. 11131D) at cells/beads ratio 1:1 in RPMI-1640 medium supplemental with 10% FBS and recombinant human IL-7 (10 ng/mL, PeproTech, Cat. No. 200-07) and IL-15 (10 ng/mL, PeproTech, Cat. No. 200-15). After 2 days activation, T cells transduce with 1 MOI lentivirus for CAR expression. On 3 days later, remove Dynabeads from transduced T cells and re-fresh medium every 2-3 days with IL-7/IL-15 (10 ng/mL, respectively) under cell density at 0.5-1×10⁶/mL. On days 10-14 after transduction, cells were collected for in vitro and in vivo experiments. T cells were cultured in IL-7- and IL-15-depleted medium for one day before being used for in vitro assays.

### Example 2. Globo H CAR-T In Vitro Cytotoxicity Assay

The cytotoxicity of Globo H CAR-T cells against Globo-H positive tumor cell line (MCF-7 and HCC-1428: breast cancer, NCI-N87: gastric carcinoma, SW480: colon cancer) and Globo-H negative tumor cell line (SK-OV-3: ovarian cancer) at serial E:T ratio (Effector CAR-T cells: Target tumor cells) in healthy donors. The luciferase stable expressed target cells pre-attached overnight in 96-well white Polystyrene plate for luminescence reading. Serial dilution of IL-7 and IL-15 depleted CAR-T cells add to target cells for 24 hours co-culture. The luminescence signal detection by Bio-Glo Luciferase Assay System (Promega, Cat. No. G7940).

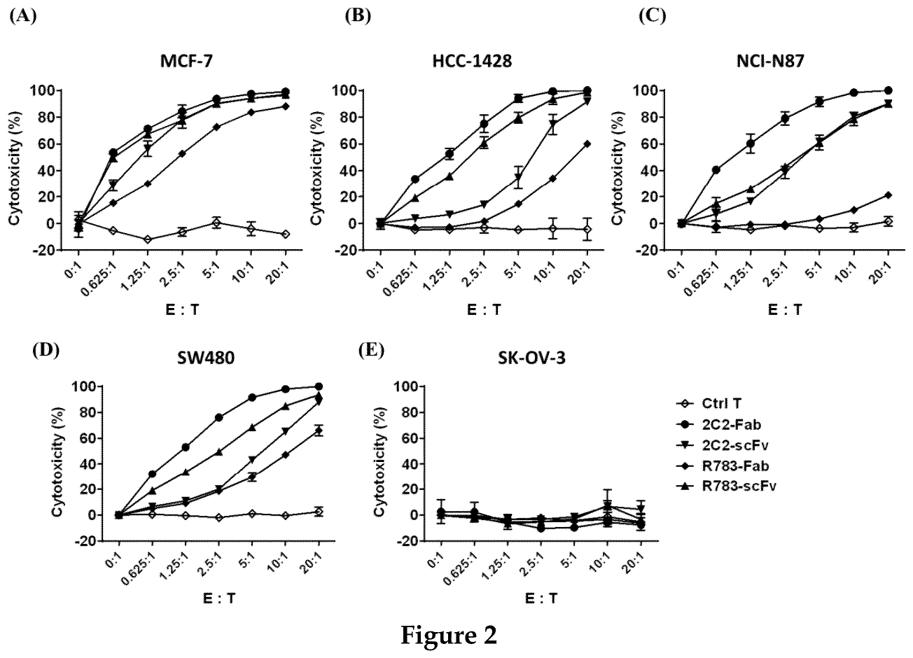

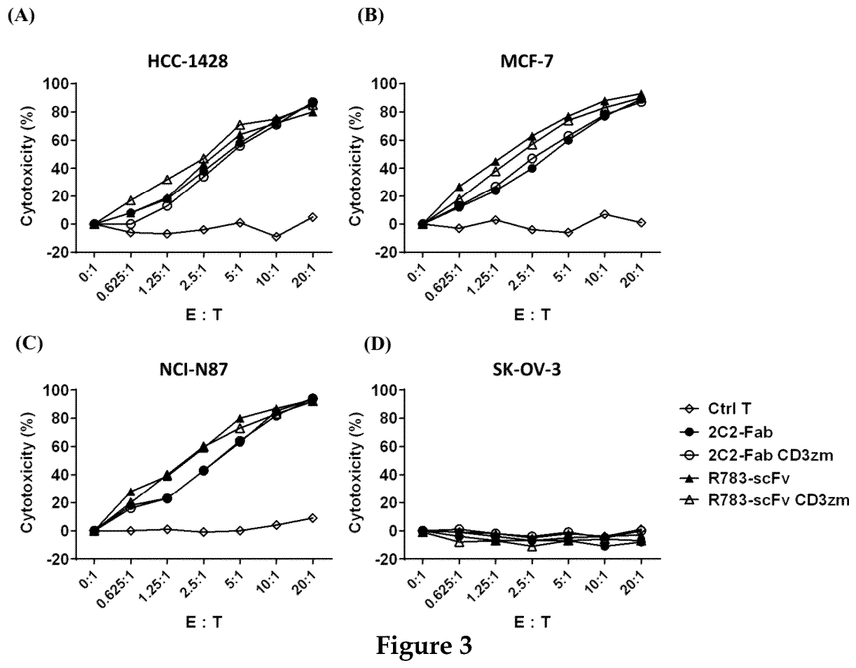

In FIG. 2, it indicated 2C2-Fab, 2C2-scFv, R783-Fab and R783-scFv CAR T cells had efficacy in Globo-H positive tumor cell lines (FIG. 2A-2D) but not existed in Globo-H negative tumor cell line (FIG. 2E). It could also demonstrate the specificity of our Globo-H CAR-T construct. In FIG. 3, it also indicated 2C2-Fab, 2C2-Fab (CD3zm), R783-scFv and R783-scFv (CD3zm) CAR T cells had similar efficacy in Globo-H positive tumor cell lines (FIG. 3A-3C) but not existed in Globo-H negative tumor cell line (FIG. 3D). It could demonstrate the CD3zeta mutant (CD3zm) CAR without loss cytotoxicity.

Furthermore, another in vitro persistence assay was performed. Luciferase reporter tumor cell lines (1×10⁵ MCF-7 or SK-OV3) were pre-stained with CellTracker™ Deep Red Dye (Thermo Fisher, Cat. No. C34565) for 15 minutes at 37° C. and then seeded in 24-well tissue culture plates for 16 hours, after which 1×10⁵ (E:T=1:1) CAR T cells were added to the tumor cells. After 3 days, tumor cells had been completely eradicated (round 1). All cells in the well were collected and washed with PBS, resuspended in fresh 2% FBS RPMI-1640 medium and added to a new plate seeded with 1×10⁵ tumor cells for 3 days (round 2). This procedure was repeated one more time, if applicable (round 3). At the end of each round, absolute cell number of residual tumor cells (APC+) and CAR T cells (CD3+) was calculated with CountBright™ Absolute Counting Beads (Thermo Fisher, Cat. No. C36950) by flow cytometry.

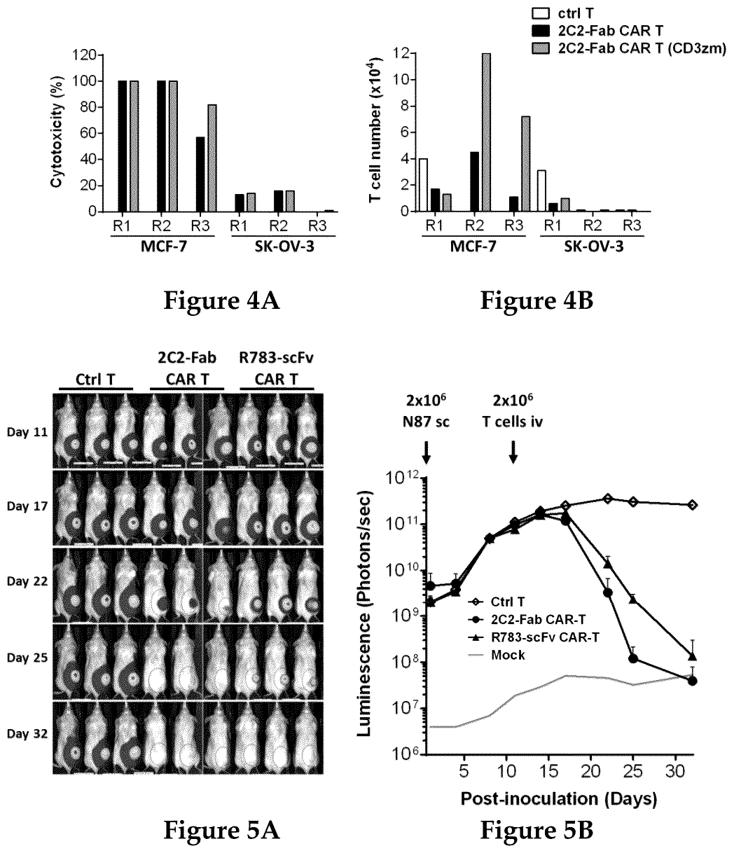

In FIG. 4, it indicated CD3zm CAR T cells have higher cytotoxicity (FIG. 4A) and more residue CAR T cells after repetitively killing assay (FIG. 4B). It could demonstrate that 2C2-Fab CAR T with CD3zeta mutant (CD3zm) is more persistent than it with wild-type CD3zeta (CD3z).

### Example 3. Globo H CAR-T In Vivo Efficacy Assay

### Example 3-1: NCI-N87 Gastric Xenograft Model

Six to eight-week-old ASID mice (NOD.Cg-Prkdc^{scid} Il2rg^{tm1Wjl}/YckNarl) were purchased from National Laboratory Animal Center (Taipei, Taiwan) and were used in all in vivo models. ASID mice were injected with 2×10⁶ N87-Luc cells mixture with Matrigel (1:1, BD Bioscience) on Day 0 by subcutaneously injection on the right flank. Mice were treated intravenously with 2×10⁶ Globo H CAR-T cells or vector control T cells at Day 11. Flank tumor size was measured in three dimensions (mm³) with calipers. Mice were imaged on an Ami-HT optical imaging system twice per week during in vivo studies, after being intraperitoneal injected with 200 μL of 15 mg/mL D-Luciferin (Biosynth, Cat. No. L-8220). Tumor volumes were calculated using the formula V=1/2 (length×width²).

In FIG. 5, it indicated 2C2-Fab and R783-scFv CAR T cells showed significant efficacy in NCI-N87 tumor model. FIG. 5A indicated representative tumor bioluminescence images from Day 11 to Day 32 post-tumor inoculation of CAR-T (n=3) and PBS control (n=3) groups. On Day 22, the overall nine ASID mice could observe tumor existed. However, after injection of 2C2-Fab or R783-scFv CAR-T cells for 14 days (Day 25) or 21 days (Day 32) respectively, there were only three ASID mice (vector control T cells) observe tumor existed. FIG. 5B indicated kinetics of tumor bioluminescence from three tumor groups and one tumor-free mock control. The curves show data for mean±SD of three mice per group.

### Example 3-2: In Vivo Efficacy of Globo H CAR T in Different Tumor Models

MCF-7 and HCC-1428 Breast Cancer Orthotopic Models:

Six to eight-week-old ASID mice were implanted subcutaneously with Estrogen pellet (0.36 mg/pellet 17 β-estradiol, 90-day release, Innovative Research of America). After two days, mice were orthotopically inoculated 8×10⁶ MCF-7 or HCC-1428 cells mixture with Matrigel (1:1, BD Bioscience) by injection at 4^{th} mammary fat pad. Mice were treated intravenously with 2×10⁶ Globo H CAR-T cells or vector control T cells at Day 12. Orthotopic tumor size was measured in three dimensions (mm 3) with calipers. Tumor volumes were calculated using the formula V=1/2 (length×width²).

SW-480 Colon Cancer Xenograft Model:

Six to eight-week-old ASID mice were injected with 1×10⁶ SW-480 cells mixture with Matrigel (1:1, BD Bioscience) on Day 0 by subcutaneously injection on the right flank. Mice were treated intravenously with 2×10⁶ Globo H CAR-T cells or vector control T cells at Day 12. Flank tumor size was measured in three dimensions (mm 3) with calipers. Tumor volumes were calculated using the formula V=1/2 (length×width²).

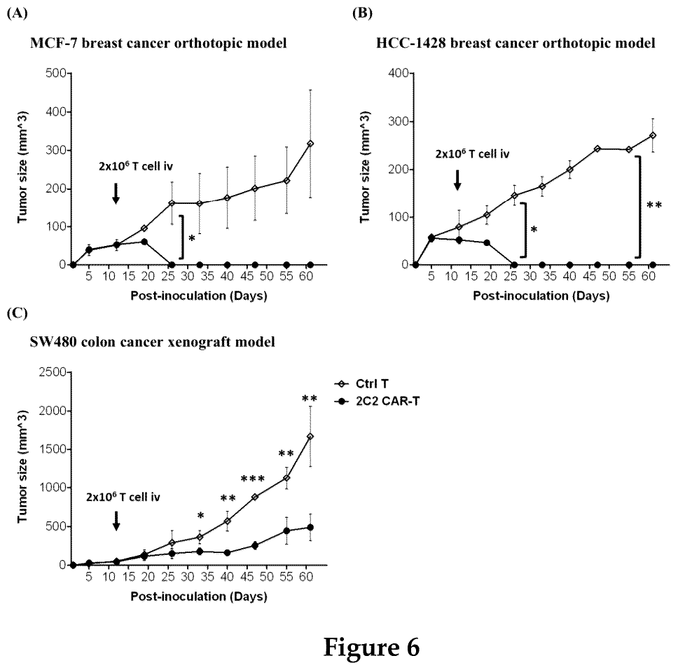

In FIG. 6, it indicated 2C2-Fab CAR T cells showed significant efficacy in MCF-7 (FIG. 6A), HCC-1428 (FIG. 6B) breast cancer orthotopic and SW-480 (FIG. 6C) colon cancer xenograft model. Kinetics of tumor size from two tumor groups injection with control T cells or 2C2-Fab CAR T cells (n=3 per group) in these models. The curves show data for mean±SD of three mice per group.

### Example 4. In Vivo Persistence of Globo H CAR T in NCI-N87 Gastric Xenograft Model

Six to eight-week-old ASID mice were injected with 2×10⁶ N87-Luc cells mixture with Matrigel (1:1, BD Bioscience) on day 0 by subcutaneously injection on the right flank. Mice were treated intravenously with 2×10⁶ Globo H CAR-T cells or vector control T cells at Day 10. For the re-challenge experiments, mice received a 2nd tumor dose 2×10⁶ of N87-Luc by subcutaneously injection on the left flank at Day 40. The 3^{rd} tumor inoculated subcutaneously with 2×10⁶ N87-Luc cells on the right flank at Day 69. Flank tumor size was measured in three dimensions (mm 3) with calipers. Mice were imaged on an Ami-HT optical imaging system twice per week during in vivo studies, after being intraperitoneal injected with 200 μL of 15 mg/mL D-Luciferin (BIOSYNTH, Cat. No. L-8220). Tumor volumes were calculated using the formula V=1/2 (length×width²).

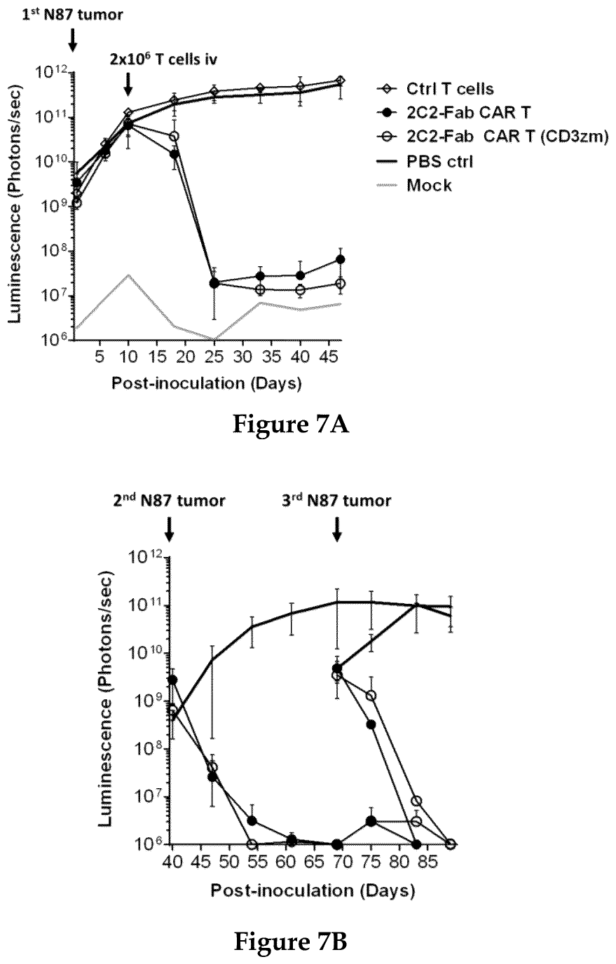

In FIG. 7, it indicated 2C2-Fab and 2C2-Fab (CD3zm) CAR T cells showed significant efficacy and persistence in NCI-N87 tumor model. FIG. 7A indicated kinetics of tumor bioluminescence in four tumor groups (1^{st} tumor growth) and one tumor-free mock control from Day 0 to Day 47. The curves show data for mean±SD of three mice per group. The primary tumors treated with 2C2-Fab CAR T or 2C2-Fab (CD3zm) CAR T cells have been all eliminated at Day 25. FIG. 7B indicated kinetics of 2nd and 3rd tumor bioluminescence in 2C2-Fab and 2C2-Fab (CD3zm) CAR T cell therapeutic groups from Day 45 to Day 90. PBS control tumor groups are tumor-free mice to inoculate with 2×10⁶ N87-Luc tumor cells as re-challenge control. The 2n d tumors have also been clearance both in 2C2-Fab CAR T or 2C2-Fab (CD3zm) CAR T cells treated groups at Day 60 (n=3 per group). Only 2C2-Fab (CD3zm) CAR T cells treated groups are all survival after 3^{rd} tumor challenge (n=3).

Unless defined otherwise, all technical and scientific terms and any acronyms used herein have the same meanings as commonly understood by one of ordinary skill in the art in the field of this invention. Although any compositions, methods, kits, and means for communicating information similar or equivalent to those described herein can be used to practice this invention, the preferred compositions, methods, kits, and means for communicating information are described herein.

All references cited herein are incorporated herein by reference to the full extent allowed by law. The discussion of those references is intended merely to summarize the assertions made by their authors. No admission is made that any reference (or a portion of any reference) is relevant prior art. Applicants reserve the right to challenge the accuracy and pertinence of any cited reference.

1. A chimeric antigen receptor (CAR), comprising:
a single-chain variable fragment (scFv) or an antigen-binding fragment (Fab) that recognizes a Globo series antigen; and
a first endodomain comprising CD3zeta or FcεRIγ,
wherein the CAR includes an amino acid sequence with 80% to 100% sequence identity to anyone of SEQ ID Nos: 13-18.

2. The CAR of claim 1, further comprises a second endodomain including CD28, CD137, CD4, OX40, 4-1BB, CD3Z, or ICOS, wherein the scFv is fused to the second endodomain, and the second endodomain is fused to the first endodomain.

3. The CAR of claim 1, wherein the scFv comprises an amino acid sequence with 80% to 100% identity to SEQ ID No: 3 or 6.

4. (canceled)

5. The CAR of claim 1, wherein the Fab comprise:
a heavy chain variable region (V_{H}) having an amino acid sequence with 80% to 100% sequence identity to SEQ ID No: 1 or 4; and
a light chain variable region (V_{L}) having an amino acid sequence with 80% to 100% sequence identity to SEQ ID No: 2 or 5.

6. The CAR of claim 1, further comprises a second endodomain including CD28, CD137, CD4, OX40, 4-1BB, CD3Z, and ICOS, wherein the Fab is fused to the second endodomain, and the second endodomain is fused to the first endodomain.

7. The CAR of claim 2, wherein the CAR comprises:
(a) a CD8 hinge region having an amino acid sequence with 90% to 100% sequence identity to SEQ ID No: 7;
(b) a CD28 endodomain sequence with 90% to 100% sequence identity to SEQ ID No: 8;
(c) a 4-1BB endodomain sequence with 90% to 100% sequence identity to SEQ ID No: 9; or
(d) a CD3zeta domain sequence with 90% to 100% sequence identity to SEQ ID No: 10 or 11.

8. The CAR of claim 6, wherein the CAR comprises:
(a) a CD8 hinge region having an amino acid sequence with 90% to 100% sequence identity to SEQ ID No: 7;
(b) a CD28 endodomain sequence with 90% to 100% sequence identity to SEQ ID No: 8;
(c) a 4-1BB endodomain sequence with 90% to 100% sequence identity to SEQ ID No: 9; or
(d) a CD3zeta domain sequence with 90% to 100% sequence identity to SEQ ID No: 10 or 11.

9. (canceled)

10. (canceled)

11. (canceled)

12. (canceled)

13. The CAR of claim 1, wherein the Globo series antigen is selected from the group consisting of Globo H, stage-specific embryonic antigen 3 (SSEA-3), and stage-specific embryonic antigen 4 (SSEA-4).

14. A method for treating a subject with a tumor, comprising:
(A) obtaining T cells from the subject having the tumor;
(B) generating chimeric antigen receptor expression T cells (CAR-T cells) by transducing the T cells with a vector comprising a nucleic acid sequence encoding a chimeric antigen receptor (CAR);
(C) expanding the CAR-T cells; and
(D) infusing the expanded CAR-T cells into the subject, whereby an immune response is raised,
wherein the CAR comprises a single-chain variable fragment (scFv) or an antigen-binding fragment (Fab) that recognizes a Globo series antigen, and the CAR comprises an amino acid sequence with 80% to 100% sequence identity to anyone of SEQ ID Nos: 13-18.

15. (canceled)

16. The method of claim 14, wherein the subject is human.

17. The method of claim 14, wherein the immune response is mediated by T cells.

18. The method of claim 14, wherein the vector comprises a lentivirus, a gamma retrovirus, or an adeno-associated vims.

19. The method of claim 14, wherein the tumor expresses Globo H.

20. The method of claim 14, wherein the tumor is selected from the group consisting of breast cancer, lung cancer, esophageal cancer, rectal cancer, biliary cancer, liver cancer, buccal cancer, gastric cancer, colon cancer, nasopharyngeal cancer, kidney cancer, prostate cancer, ovarian cancer, cervical cancer, endometrial cancer, pancreatic cancer, testicular cancer, bladder cancer, head and neck cancer, oral cancer, neuroendocrine cancer, adrenal cancer, thyroid cancer, bone cancer, skin cancer, basal cell carcinoma, squamous cell carcinoma, melanoma, and brain tumor.

21. The method of claim 14, wherein the CAR further comprises a first endodomain including CD3zeta or FcεRIγ.

22. The method of claim 14, wherein the CAR further comprises a second endodomain including CD28, CD137, CD4, OX40, 4-1BB, CD3Z, or ICOS.

23. The method of claim 14, wherein the CAR further comprises a hinge region of CD8.

24. The method of claim 14, wherein the Globo series antigen is selected from the group consisting of Globo H, stage-specific embryonic antigen 3 (SSEA-3), and stage-specific embryonic antigen 4 (S SEA-4).


SEQUENCE LISTING (18 sequences)

SEQ ID NO: 1 - protein, 243 aa, Artificial Sequence
  2C2 Heavy Chain (HC)
MDMRVPAQLLGLLLLWLPGAKCQVTLKESGPGILQPSQTLSLTCSFSGFSLYTFDMGVGWIRQPSGKGLEWLAHIWWDDDKYYNPALKSRLTVSKDTSKNQVFLKIPNVDTADSATYYCARVRGLHDYYYWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 2 - protein, 235 aa, Artificial Sequence
  2C2 Light Chain (LC)
MDMRVPAQLLGLLLLWLPGAKCQIVLSQSPTILSASPGEKVTMTCRASSSVSYMHWYQQKPGSSPKPWIYATSNLASGVPARFSGSGSGTSYSLTISRVEAEDAATYFCQQWSRNPFTFGQGTKVEIKRTVAAPSVFIFPPSDEQLKSGTASVVCLLNNFYPREAKVQWKVDNALQSGNSQESVTEQDSKDSTYSLSSTLTLSKADYEKRKVYACEVTHQGLSSPVTKSFNRGEC

SEQ ID NO: 3 - protein, 272 aa, Artificial Sequence
  2C2 scFv
MDFQVQIFSFLLISASVIMSRLPGAKCQVTLKESGPGILQPSQTLSLTCSFSGFSLYTFDMGVGWIRQPSGKGLEWLAHIWWDDDKYYNPALKSRLTVSKDTSKNQVFLKIPNVDTADSATYYCARVRGLHDYYYWFAYWGQGTLVTVSSGGGGSGGGGSGGGGSQIVLSQSPTILSASPGEKVTMTCRASSSVSYMHWYQQKPGSSPKPWIYATSNLASGVPARFSGSGSGTSYSLTISRVEAEDAATYFCQQWSRNPFTFGQGTKVEIKR

SEQ ID NO: 4 - protein, 241 aa, Artificial Sequence
  R783 Heavy Chain (HC)
METDTLLLWVLLLWVPGSTGQITLQESGPTLVKPTQTLTLTCTFSGFSLYRFDMGVGWIRQPPGQGLEWLAHIWWDDDKYYNPALKSRLTISKDTSKNQVVLTMTNMDPVDTATYYCARVRGLHDYYYYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 5 - protein, 233 aa, Artificial Sequence
  R783 Light Chain (LC)
METDTLLLWVLLLWVPGSTGEIVLTQSPSTLSLSPGERATLSCQASEDVSYMHWYQQKPGQAPQPWIYGTSNKASGVPSRFSGSGSGTDFTLTISSLQPEDVATYYCQQWSRRPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 265 aa, Artificial Sequence
  R783 scFv
METDTLLLWVLLLWVPGSTGQITLQESGPTLVKPTQTLTLTCTFSGFSLYRFDMGVGWIRQPPGQGLEWLAHIWWDDDKYYNPALKSRLTISKDTSKNQVVLTMTNMDPVDTATYYCARVRGLHDYYYYFAYWGQGTLVTVSSGGGGSGGGGSGGGGSEIVLTQSPSTLSLSPGERATLSCQASEDVSYMHWYQQKPGQAPQPWIYGTSNKASGVPSRFSGSGSGTDFTLTISSLQPEDVATYYCQQWSRRPFTFGQGTKVEIKR

SEQ ID NO: 7 - protein, 45 aa, Artificial Sequence
  CD8 Hinge
TTTPAPRPPTPAPTIASQPLSLRPEACRPAAGGAVHTRGLDFACD

SEQ ID NO: 8 - protein, 68 aa, Artificial Sequence
  CD28 transmembrane & costimulatory domain
FWVLVVVGGVLACYSLLVTVAFIIFWVRSKRSRLLHSDYMNMTPRRPGPTRKHYQPYAPPRDFAAYRS

SEQ ID NO: 9 - protein, 42 aa, Artificial Sequence
  4-1BB costimulatory domain
KRGRKKLLYIFKQPFMRPVQTTQEEDGCSCRFPEEEEGGCEL

SEQ ID NO: 10 - protein, 114 aa, Artificial Sequence
  CD3zeta domain
LRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 11 - protein, 114 aa, Artificial Sequence
  CD3zeta mutant (CD3zm) domain
LRVKFSRSADAPAYQQGQNQLYNELNLGRREEYDVLDKRRGRDPEMGGKPQRRKNPQEGLFNELQKDKMAEAFSEIGMKGERRRGKGHDGLFQGLSTATKDTFDALHMQALPPR

SEQ ID NO: 12 - protein, 21 aa, Artificial Sequence
  T2A linker
GSGEGRGSLLTCGDVEENPGP

SEQ ID NO: 13 - protein, 768 aa, Artificial Sequence
  2C2-Fab CAR
MDMRVPAQLLGLLLLWLPGAKCQVTLKESGPGILQPSQTLSLTCSFSGFSLYTFDMGVGWIRQPSGKGLEWLAHIWWDDDKYYNPALKSRLTVSKDTSKNQVFLKIPNVDTADSATYYCARVRGLHDYYYWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGEGRGSLLTCGDVEENPGPMDMRVPAQLLGLLLLWLPGAKCQIVLSQSPTILSASPGEKVTMTCRASSSVSYMHWYQQKPGSSPKPWIYATSNLASGVPARFSGSGSGTSYSLTISRVEAEDAATYFCQQWSRNPFTFGQGTKVEIKRTVAAPSVFIFPPSDEQLKSGTASVVCLLNNFYPREAKVQWKVDNALQSGNSQESVTEQDSKDSTYSLSSTLTLSKADYEKRKVYACEVTHQGLSSPVTKSFNRGEC

SEQ ID NO: 14 - protein, 541 aa, Artificial Sequence
  2C2-scFv CAR
MDFQVQIFSFLLISASVIMSRLPGAKCQVTLKESGPGILQPSQTLSLTCSFSGFSLYTFDMGVGWIRQPSGKGLEWLAHIWWDDDKYYNPALKSRLTVSKDTSKNQVFLKIPNVDTADSATYYCARVRGLHDYYYWFAYWGQGTLVTVSSGGGGSGGGGSGGGGSQIVLSQSPTILSASPGEKVTMTCRASSSVSYMHWYQQKPGSSPKPWIYATSNLASGVPARFSGSGSGTSYSLTISRVEAEDAATYFCQQWSRNPFTFGQGTKVEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 15 - protein, 764 aa, Artificial Sequence
  R783-Fab CAR
METDTLLLWVLLLWVPGSTGQITLQESGPTLVKPTQTLTLTCTFSGFSLYRFDMGVGWIRQPPGQGLEWLAHIWWDDDKYYNPALKSRLTISKDTSKNQVVLTMTNMDPVDTATYYCARVRGLHDYYYYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGEGRGSLLTCGDVEENPGPMETDTLLLWVLLLWVPGSTGEIVLTQSPSTLSLSPGERATLSCQASEDVSYMHWYQQKPGQAPQPWIYGTSNKASGVPSRFSGSGSGTDFTLTISSLQPEDVATYYCQQWSRRPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 534 aa, Artificial Sequence
  R783-scFv CAR
METDTLLLWVLLLWVPGSTGQITLQESGPTLVKPTQTLTLTCTFSGFSLYRFDMGVGWIRQPPGQGLEWLAHIWWDDDKYYNPALKSRLTISKDTSKNQVVLTMTNMDPVDTATYYCARVRGLHDYYYYFAYWGQGTLVTVSSGGGGSGGGGSGGGGSEIVLTQSPSTLSLSPGERATLSCQASEDVSYMHWYQQKPGQAPQPWIYGTSNKASGVPSRFSGSGSGTDFTLTISSLQPEDVATYYCQQWSRRPFTFGQGTKVEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 17 - protein, 768 aa, Artificial Sequence
  2C2-Fab CAR (CD3zm)
MDMRVPAQLLGLLLLWLPGAKCQVTLKESGPGILQPSQTLSLTCSFSGFSLYTFDMGVGWIRQPSGKGLEWLAHIWWDDDKYYNPALKSRLTVSKDTSKNQVFLKIPNVDTADSATYYCARVRGLHDYYYWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELLRVKFSRSADAPAYQQGQNQLYNELNLGRREEYDVLDKRRGRDPEMGGKPQRRKNPQEGLFNELQKDKMAEAFSEIGMKGERRRGKGHDGLFQGLSTATKDTFDALHMQALPPRGSGEGRGSLLTCGDVEENPGPMDMRVPAQLLGLLLLWLPGAKCQIVLSQSPTILSASPGEKVTMTCRASSSVSYMHWYQQKPGSSPKPWIYATSNLASGVPARFSGSGSGTSYSLTISRVEAEDAATYFCQQWSRNPFTFGQGTKVEIKRTVAAPSVFIFPPSDEQLKSGTASVVCLLNNFYPREAKVQWKVDNALQSGNSQESVTEQDSKDSTYSLSSTLTLSKADYEKRKVYACEVTHQGLSSPVTKSFNRGEC

SEQ ID NO: 18 - protein, 534 aa, Artificial Sequence
  R783-scFv CAR (CD3zm)
METDTLLLWVLLLWVPGSTGQITLQESGPTLVKPTQTLTLTCTFSGFSLYRFDMGVGWIRQPPGQGLEWLAHIWWDDDKYYNPALKSRLTISKDTSKNQVVLTMTNMDPVDTATYYCARVRGLHDYYYYFAYWGQGTLVTVSSGGGGSGGGGSGGGGSEIVLTQSPSTLSLSPGERATLSCQASEDVSYMHWYQQKPGQAPQPWIYGTSNKASGVPSRFSGSGSGTDFTLTISSLQPEDVATYYCQQWSRRPFTFGQGTKVEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELLRVKFSRSADAPAYQQGQNQLYNELNLGRREEYDVLDKRRGRDPEMGGKPQRRKNPQEGLFNELQKDKMAEAFSEIGMKGERRRGKGHDGLFQGLSTATKDTFDALHMQALPPR